(19) United States
(12) Patent Application Publication
(10) Pub. No.: US 2017/0296398 A1
(43) Pub. Date: Oct. 19, 2017

(54) Incontinence Garment

(71) Applicant: William Frazier, JR., Newark, NJ (US)

(72) Inventor: William Frazier, JR., Newark, NJ (US)

(21) Appl. No.: 15/418,604

(22) Filed: Jan. 27, 2017

Related U.S. Application Data
(60) Provisional application No. 62/287,703, filed on Jan. 27, 2016.

(51) Int. Cl. A61F 13/496; A61F 13/49; A61F 13/491; A61F 13/72

(52) U.S. Cl. CPC A61F 13/496; A61F 13/4915; A61F 13/72; A61F 13/49011; A61F 13/491; A61F 2013/49088

(57) ABSTRACT

An incontinence garment for wear by a user is provided. The user has an anatomy. The incontinence garment comprises a protective undergarment having a waist opening and a pair of leg openings. An opening is formed in the protective undergarment approximately centered between the leg openings for accommodating the wearers anatomy. An outer garment is positionable over the protective undergarment with the outer garment having a waist opening and a pair leg openings and the outer garment being free from any other openings. The combined protective undergarment and outer garment offers a dual layer of absorbent protection.

### CLAIM OR PRIORITY

This patent application claims priority under 35 USC 119 (e) (1) from U.S. Provisional Patent Application Ser. No. 62/287,703 filed Jan. 27, 2016, of common inventorship herewith entitled, “Pee Dry,” which is incorporated herein by reference as though the same were set forth in its entirety.

### FIELD OF THE INVENTION

The present invention pertains to the field of urine incontinence, and more specifically to the field of absorbent garments, including diapers or underwear

### BACKGROUND OF THE INVENTION

As a result of illness or age, many people are effected by incontinence. The inability to control one's bodily functions can diminish one's confidence as well as limits one's ability to freely function in society. The best way to control incontinence symptoms is by wearing protective undergarments such as sanitary liners or disposable underwear. Manufactured of sanitary paper material, these protective products are constructed to provide individuals with a simple and hygienic means of absorbing excess bodily fluids, preventing embarrassing stains while safeguarding expensive clothing. While sanitary liners and protective underwear are indeed practical those persons who suffer more serious cases of loss of bladder control find they must repeatedly change these garments throughout the day to stay comfortable and dry.

Incontinent issues also are quite common with infants and toddlers learning to use a commode. As a parent of an infant child learns, constant diaper changes are simply a fact of life until the child learns to control their bladder and is fully trained to use a commode Incontinence diapers are expensive and constant diaper changes are time consuming and add up to a serious depletion of one's pocket book.

The prior art has put forth several designs for absorbent diapers or underwear. Among these are:

U.S. Pat. No. 7,658,730 to N. Sharon Conley describes an external male urine collection device inserted into a front opening of a disposable nonwoven diaper or brief. The device comprises a resilient ring for receiving and restraining the penis in a funnel shaped collection assembly bag extending from the diaper or brief. A distal funnel like end of the collection bag is attached to a one way valve and tube assembly to direct the urine from the bag. The tube is attached to a bedside urine storage bag or leg bag

U.S. Pat. No. 5,462,541 to Mary A. Bruemmer, Paulette M. Rosch, John P. Vukos, Dwayne G. Uitenbroek, Georgia L. Zehner and Christine A. Rasmussen describes a diaper or absorbent article comprising a top sheet a back sheet art absorbent between the top sheet and back sheet, and an elevating device for spacing the top sheet above the absorbent to form a pocket shape. The top sheet has an opening and the absorbent has a hole therein, both of which receive and isolate waste material from the wearer.

U.S. Pat. No. 5,062,840 to John Holt and Debra S. Holt describes an improved diaper to allow fecal materials to be retained away from the body. It includes a water impervious outer layer, a water retention or absorption intermediate layer and an inner or inside layer which is water pervious. The inner layer has an anal opening for waste passage. Immediately below the anal opening in the intermediate layer is an oval shaped solids cavity. Send waste material from the baby is deposited in this cavity and extends in the space between the intermediate layer and the outer layer. The outer layer has a pleat which is aligned with the solids cavity to give more room for waste products. A water soluble cover, which disintegrates in water, is provided for the solids cavity of the intermediate layer.

U.S. Pat. No. 4,695,279 to Graham E. Steer describes a pair of incontinence pants or briefs that includes a pocket located in the crotch region. The pocket is defined by an outer wall of a liquid impermeable material that is longer than it is wide and an inner wall of like material whose edges define a substantially oval hole. A portion of the inner wall overlaps, but is not connected to another thereof and the edges are crossed over one another at one end of the pocket.

None of these prior art references describe the present invention.

### SUMMARY OF THE INVENTION

It is an object of the present invention to provide a line of specially constructed incontinence garments comprising a dual layer of absorbent protection.

The present invention is an incontinence garment tor wear by a user. The user has an anatomy. The incontinence garment comprises a protective undergarment having a waist opening and a pair of leg openings. An opening is formed in the protective undergarment approximately centered between the leg openings for accommodating the wearers anatomy. An outer garment is positional over the protective undergarment with the outer garment having a waist opening and a pair leg openings and the outer garment being free from any other openings. The combined protective undergarment and outer garment offers a dual layer of absorbent protection.

In addition, the present invention includes a method for constructing an incontinence garment for wear by a user. The user has an anatomy. The method comprises providing a protective undergarment having a waist opening and a pair of leg opening, forming an opening in the protective undergarment approximately centered between the leg openings for accommodating the wearer's anatomy, positioning an outer garment over the protective undergarment with the outer garment having a waist opening and a pair of leg openings and the outer garment being free from any other openings, and offering the user a dual layer of absorbent protection.

The present invention further includes an incontinence garment for wear by a user. The user has an anatomy. The incontinence garment comprises a protective undergarment having a self-sealing waist opening and a pair of self-sealing leg openings. An opening is formed in the protective undergarment approximately centered between the leg openings for accommodating the wearer's anatomy. An outer garment is positionable over the protective undergarment with the outer garment having a self-sealing waist opening and a pair of self-sealing leg openings and the outer garment being free from any other openings. The combined protective undergarment and outer garment offers a dual layer of absorbent protection.

### BRIEF DESCRIPTION OF THE DRAWINGS

FIG. 1 is a perspective view illustrating a male version of an incontinence garment, constructed in accordance with the present invention, showing an opening constructed for male anatomy.

FIG. 2 is a perspective view illustrating a female version of the incontinence garment, constructed in accordance with the present invention showing an opening constructed for female anatomy.

FIG. 3 is a perspective view illustrating the female version of the incontinence garment, constructed in accordance with the present invention, showing the incontinence garment wearable inside diapers.

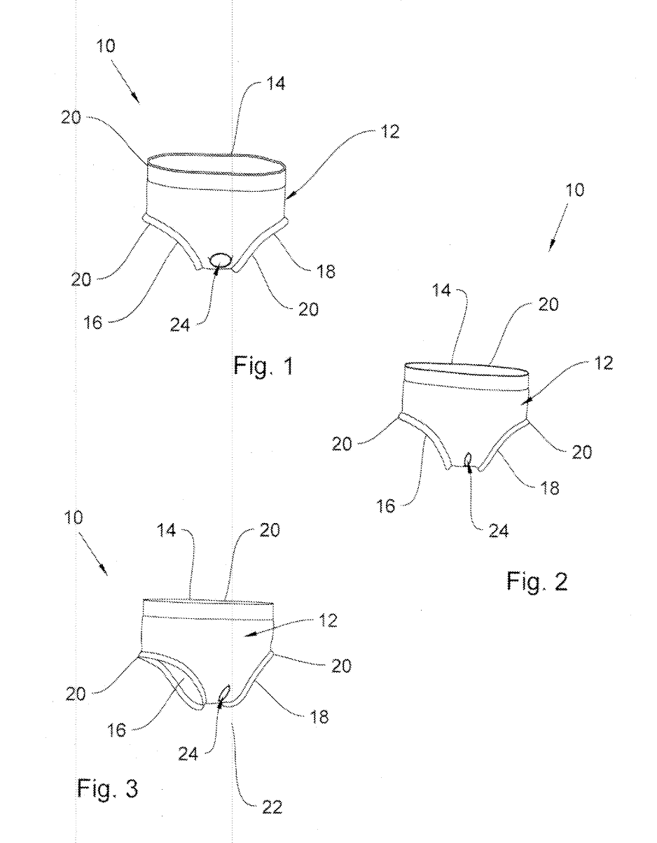

### DETAILED DESCRIPTION OF THE INVENTION

The present invention, hereinafter referred to as an Incontinence Garment, indicated generally at 10, is a line of specially constructed incontinence underwear comprising a dual layer of absorbent protection. As a protective undergarment 12, the incontinence Garment 10 provides significantly longer wear than current diapers, thus sparing a wearer repeated changes throughout the day. The protective undergarment 12 of the incontinence Garment 10 is manufactured in a range of sizes appropriate for wear by adults and children.

The Incontinence Garment 10 of the present invention includes the protective undergarment 12 having a waist opening 14 and a pair of leg openings 18, 18. Preferably, the waist opening 14 and the leg openings 18, 18 include elastic banding 20 completely about the waist opening 14 and the leg openings 16, 18 for facilitating a comfortable and customized fit. In an embodiment, the protective undergarment 12 has a one piece elastic waistband that is pulled up and over the body in the same manner as traditional underwear or briefs. In another embodiment, rather than an elastic waistband, the waist opening 14 of the protective undergarment 12 is secured about the waist my means of tab fasteners positioned over each hip portion.

The protective undergarment 12 of the Incontinence Garment 10 of the present invention is preferably manufactured of a super absorbent sanitary paper material layer having a soft plastic backing to prevent leaks. The protective undergarments 12 from the Incontinence Garment 10 line are produced in different versions for wear by male and females. Each version of the Incontinence Garment 10 is comprised of a form fitted under layer over which a second sanitary garment 22 is applied.

In addition, the protective undergarment 12 of the Incontinence Garment 10 of the present invention includes an opening 24 formed approximately between the leg openings 16, 18 of the protective undergarment 12 that accommodates the wearer's anatomy. In the case of protective undergarments 12 for wear by men, the opening 24 is a substantially round or oval opening positioned at the front of the protective undergarment 12 to accommodate a penis. In the case of protective undergarments 12 for wear by females have an oval or elongated opening that encompasses a crotch panel of the protective undergarments 12 over a female's vaginal area and urethral opening.

The super absorbent secondary garment or outer layer 22 of the Incontinence Garment 10 of the present invention that is included for wear with the aforementioned protective undergarment 12 is similar in appearance to a traditional adult or child's diaper. The outer layer 22 is worn over the protective undergarment 12. The outer layer 22 is secured about the waist by means of a solid pull up banding or by means of self-affixing fasteners that are secured at each hip of the user.

The Incontinence Garment 10 of the present invention provides individuals who suffer incontinence with a practical alternative to traditional sanitary undergarments and drapers and enable a wearer to stay dry and comfortable for extended periods of time. Comprising two layers of protection, the Incontinence Garment 10 pulls moisture away from a body and into the outer layer of the garment enabling air to freely circulate about the body. Enabling a wearer to maintain a feeling of being dry and fresh for longer periods, wearing the Incontinence Garment 10 significantly reduces the amount of diaper changes needed throughout the day, saving a wearer their caregiver an unpleasant task of constantly changing one's diaper. Absorbing moisture and preventing unsightly and embarrassing leaks and stains from occurring, using the Incontinence Garment 10 allows wearers supreme comfort, regardless of activity levels or bladder control issues. Durably constructed with high quality materials, the Incontinence Garment 10 will permit wearers to enjoy an active lifestyle with confidence and peace of mind.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. An incontinence garment for wear by a user, the user having an anatomy, the incontinence garment comprising:
a protective undergarment having a waist opening and a pair of leg openings;
an opening formed in the protective undergarment approximately centered between the leg openings for accommodating the wearers anatomy; and
an outer garment positionable over the protective undergarment, the outer garment having a waist opening end a pair leg openings, the outer garment being free from any other openings;
wherein the combined protective undergarment and outer garment offers a dual layer of absorbent protection.

2. The incontinence garment of claim 1 wherein the waist opening and leg openings of the protective undergarment include elastic banding comply about the waist opening and the leg openings of the protective undergarment.

3. The incontinence garment of claim 2 wherein elastic banding of the protective undergarment is a one piece elastic waistband.

4. The incontinence garment of claim 1 wherein the waist opening of the protective undergarment has tab fasteners.

5. The incontinence garment of claim 1 wherein the waist opening and leg openings of the outer garment include elastic banding completely about the waist opening and the leg openings of the outer garment.

6. The incontinence garment of claim 1 wherein the protective undergarment Is constructed from an absorbent sanitary paper material layer having a soft plastic backing.

7. The incontinence garment of claim 1 wherein the protective undergarment is a male undergarment and the opening is a substantially round or oval opening positioned at the front of the garment

8. The incontinence garment of claim 1 wherein the protective undergarment is a female undergarment and the opening is an oval or elongated opening positioned at a crotch panel of the protective undergarment.

9. A method for constructing an incontinence garment for wear by a user, the user having an anatomy, the method comprising:
providing a protective undergarment having a waist opening and a pair of leg openings:
forming an opening m the protective undergarment approximately centered between the leg openings tor accommodating the wearer's anatomy:
positioning an outer garment over the protective undergarment, the outer garment having a waist opening and a pair of leg openings, the outer garment being free from any other openings; and
offering the user a dual layer of absorbent protection.

10. The method of claim 9 and further comprising:
positioning elastic banding completely around the waist opening and leg openings of the protective undergarment.

11. The method of claim 10 wherein elastic banding of the protective undergarment is a one piece elastic waistband.

12. The method of claim 9 and further comprising:
mounting tab fasteners about the waist opening of the protective undergarment.

13. The method of claim 9 and further comprising:
mounting elastic banding completely about the waist opening and the leg openings of the outer garment.

14. The method of claim 9 and further comprising:
constructing the protective undergarment constructed from an absorbent sanitary paper material layer having a soft plastic backing.

15. The method of claim 9 wherein the protective undergarment is a male undergarment and further comprising:
forming the opening as a substantially round or oval opening; and
positioning the opening at the front of the garment.

16. The method of claim 9 wherein the protective undergarment is a female undergarment and further comprising:
forming the opening as an oval or elongated opening, and
positioning the opening at a crotch panel of the protective undergarment.

17. An incontinence garment for wear by a user, the user having an anatomy, the incontinence garment comprising:
a protective undergarment having a self-sealing waist opening and a pair of self-sealing leg openings;
an opening formed in the protective undergarment approximately centered between the leg openings for accommodating the wearer's anatomy; and
an outer garment positionable over the protective undergarment, the outer garment having a self-sealing waist opening and a pair of self-sealing leg openings, the outer garment being free from any other openings;
wherein the combined protective undergarment and outer garment offers a dual layer of absorbent protection.

18. The incontinence garment of claim 16 wherein the waist opening and leg openings of the protective undergarment and the waist opening and leg openings of the outer garment include elastic banding completely about the waist openings and the leg openings.

19. The incontinence garment of claim 16 wherein the protective undergarment is a male undergarment and the opening is a substantially round or oval opening positioned at the front of the garment.

20. The incontinence garment of claim 16 wherein the protective undergarment is a female undergarment and the opening is an oval or elongated opening positioned at a crotch panel of the protective undergarment.
